(19) United States
(12) Patent Application Publication
(10) Pub. No.: US 2015/0247126 A1
(43) Pub. Date: Sep. 3, 2015

(54) METHOD OF CULTURING INDUCED PLURIPOTENT STEM CELL AND MATERIAL FOR CULTURING THE SAME

(71) Applicants: NATIONAL UNIVERSITY CORPORATION KYUSHU UNIVERSITY, Fukuoka-shi (JP); NATIONAL UNIVERSITY CORPORATION KYOTO UNIVERSITY, Kyoto (JP)

(72) Inventors: Satoru KIDOAKI, Fukuoka-shi (JP); Ayaka UTSUMI, Fukuoka-shi (JP); Koji ETO, Kyoto-shi (JP)

(21) Appl. No.: 14/459,017

(22) Filed: Aug. 13, 2014

(30) Foreign Application Priority Data
Feb. 28, 2014 (JP) 2014-039524

(51) Int. Cl. C12N 5/074

(52) U.S. Cl. CPC C12N 5/0696; C12N 2501/58; C12N 2501/998

(57) ABSTRACT

A method of culturing induced pluripotent stem cells includes using a cell culture substrate including an elasticity-variable gel having an elastic modulus larger than 1 kPa and smaller than 100 MPa and a cell adhesion protein immobilized on a surface of the elasticity-variable gel.

### INCORPORATION BY REFERENCE

The disclosure of Japanese Patent Application No. 2014-039524 filed on Feb. 28, 2014 including the specification, drawings and abstract is incorporated herein by reference in its entirety.

### BACKGROUND OF THE INVENTION

1. Field of the Invention

The present invention relates to a method and a material for culturing induced pluripotent stem cells while their sternness is maintained, and in particular to a method and a material that enable rapid growth of the induced pluripotent stem cells with suppressed differentiation without using feeder cells, which have been used in conventional culture methods.

2. Description of Related Art

An induced pluripotent stem cell (hereinafter also called iPS cell) can be obtained by inducing four genes, Oct3/4, Sox2, Klf4, and c-Myc, into a somatic cell to initialize the somatic cell. iPS cells have differentiation potential to three kinds of germ layers that is equivalent to that of embryonic stem cells (called ES cells). An existing important task is to cause rapid growth of a large number of iPS cells maintained in undifferentiated state.

General culture methods that have been established for iPS cells include a method using mouse or human cellular components (using a feeder cell sheet) and a method using a living biological (mouse) raw material (using BD Matrigel (registered trademark), which is a feeder-free method). These methods strongly depend on biological properties of a culture substrate.

On the other hand, it has been known that the surface chemical properties and mechanical properties of a cell culture substrate are important factors involved in not only speeding-up (increase in the efficiency and growth rate) of the cell culture but also lineage determination on stem cell differentiation. For example, a cell culture apparatus including an elastomer having Young's modulus in a certain range (Patent Document 1), and a method of introducing differentiation of a cluster of mesenchymal stem cells, the method using a coated polyacrylamide gel having a rigidity in a certain range (Patent Document 2).

### PRIOR ART DOCUMENTS

Patent documents

Patent Document 1: Japanese Patent Application Publication No. 2009-540805 (JP 2009-540805 A)

Patent Document 2: Patent Application Publication No. 2010-532166 (JP 2010-532166 A)

The methods that mainly use biological cellular materials, such as methods using feeder cells or Matrigel as described above, can involve contamination by another protein or other substances and latent harmful pathogens, so that the safety of such materials is uncertain inside a human body. Thus, it cannot be said that these methods are suitable as culture methods for clinical application of iPS cells to humans.

In addition, although it is important to examine and define the surface chemical properties and mechanical properties of the culture substrate in culturing iPS cells, important parameters in view of such chemical or physical properties have not been sufficiently examined in designing the culture substrate used in culturing iPS cells.

### SUMMARY OF THE INVENTION

The present invention is directed to establish a design guide of a substrate for controlling undifferentiation/differentiation of iPS cells by use of an elastic substrate that makes it possible to systematically design surface chemical properties and mechanical properties which can be important factors in differentiation of iPS cells.

That is, the present invention provides a culture substrate that eliminates the use of feeder cells essential in conventional typical methods of culturing iPS cells, and particularly provides a culture method that enables rapid growth of iPS cells with various examinations of the culture substrate in view of the surface chemical properties and elastic modulus thereof.

As a result of intensive study in order to achieve the objects, the inventors of the present invention have found that rapid grown of iPS cells can be achieved with a culture substrate of an elasticity-variable gel having a certain surface elastic modulus with cell adhesion proteins immobilized on its surface, and consequently completed the present invention.

Specifically, the present invention relates to: according to a first aspect, a method of culturing an induced pluripotent stem cell, the method comprising using a cell culture substrate including an elasticity-variable gel having an elastic modulus larger than 1 kPa and smaller than 100 MPa and a cell adhesion protein immobilized on a surface of the elasticity-variable gel;

according to a second aspect, the culture method of the first aspect in which the elasticity-variable gel is a gel of a photocurable gelatin; according to a third aspect, the culture method of the second aspect in which the photocurable gelatin is a photocurable styrenated gelatin;

according to a fourth aspect, the culture method of the first aspect in which the cell adhesion protein is a laminin;

according to a fifth aspect, the culture method of the fourth aspect in which the laminin is laminin-111;

according to a sixth aspect, the culture method of any one of the first to fifth aspects in which the cell adhesion protein is immobilized at a reacting concentration of 0.1 mg to 2.0 mg with respect to 1 mL of the elasticity-variable gel;

according to a seventh aspect, the culture method of any one of the first to sixth aspects in which the induced pluripotent stem cell is a human induced pluripotent stem cell;

according to an eighth aspect, a culture substrate for induced pluripotent stem cells, the culture substrate comprising an elasticity-variable gel having an elastic modulus larger than 1 kPa and smaller than 100 MPa and a cell adhesion protein immobilized on a surface the elasticity-variable gel;

according to a ninth aspect, the culture substrate of the eighth aspect in which the elasticity-variable gel is a gel of a photocurable gelatin;

according to a tenth aspect, the culture substrate of the ninth aspect in which the photocurable gelatin is a photocurable styrenated gelatin;

according to an eleventh aspect, the culture substrate of the eighth aspect in which the cell adhesion protein is a laminin;

according to a twelfth aspect, the culture substrate of the eleventh aspect in which the laminin is laminin-111;

according to a thirteenth aspect, the culture substrate of any one of the eighth to twelfth aspects in which the cell adhesion protein is immobilized at a reacting concentration of 0.1 mg to 2.0 mg with respect to 1 mL of the elasticity-variable gel;

according to a fourteenth aspect, the culture substrate of any one of the eighth to thirteenth aspects in which the induced pluripotent stem cell is a human induced pluripotent stem cell;

according to a fifteenth aspect, a gel comprising a photocurable gelatin and a laminin immobilized on a surface of the gel;

according to a sixteenth aspect, the gel of the fifteenth aspect in which the photocurable gelatin is a photocurable styrenated gelatin; and according to a seventeenth aspect, the gel of the fifteenth aspect in which the laminin is laminin-111.

The culture method of the present invention can eliminate the use of feeder cells, which have been conventionally used in culturing iPS cells, and can achieve efficient growth and culture of iPS cells while their sternness (retaining the self-replicating growth potential, maintaining the undifferentiation property, and retaining the multipotency) is maintained

### BRIEF DESCRIPTION OF THE DRAWINGS

FIG. 1 is a graph illustrating change in surface elastic moduli (Young's moduli) of a photocurable gelatin gel prepared in Production Example 1 with respect to the irradiation time of light during gel preparation;

FIGS. 2A and 2B are graphs illustrating the amount of laminins immobilized on a gelatin gel prepared in Production Example 2, evaluated by immunofluorescent staining, where FIG. 2A illustrates change in fluorescence intensity with respect to the concentration of reacted laminins and FIG. 2B illustrates change in fluorescence intensity in 14 days after the laminin immobilization;

FIG. 3 shows phase-contrast microscopic pictures showing adhesion and growth of iPS cells cultured for 7 days on a culture substrate (3-kPa substrate) in Design Example 1;

FIG. 4 shows phase-contrast microscopic pictures showing adhesion and growth of iPS cells cultured for 7 days on a culture substrate (20-kPa substrate) in Design Example 2;

FIG. 5 shows phase-contrast microscopic pictures showing adhesion and growth of iPS cells cultured for 7 days on a culture substrate (50-kPa substrate) in Design Example 3;

FIG. 6 shows phase-contrast microscopic pictures showing adhesion and growth of iPS cells cultured for 7 days on a culture substrate (100-kPa substrate) in Design Example 4;

FIG. 7 shows phase-contrast microscopic pictures showing adhesion and growth of iPS cells cultured for 7 days on a culture substrate (Matrigel (registered trademark)) in Comparative Example;

FIGS. 8A to 8D show graphs illustrating growth curves of iPS cells cultured on culture substrates in Design Examples 1 to 4 and Comparative Example (change in density of immobilized cells at different LAM-reaction concentrations with respect to culturing days), and show results of the 3-kPa substrates, the 20-kPa substrates, the 50-kPa substrates and the Matrigel substrate, and the 100-kPa substrates and the Matrigel substrate, respectively;

FIGS. 9A to 9D show images of immunofluorescent-stained undifferentiation marker proteins observed in iPS cells that were cultured for seven days on the culture substrate in Example 2 (Design Example 5) and then retrieved to be reseeded and cultured on mouse embryonic fibroblasts (MEFs), and show SSEA-4, TRA-1-60, TRA-1-81, and SSEA-1, respectively;

FIGS. 10A to 10C show images of immunofluorescent-stained triploblastic differentiation marker proteins in an embryoid that was prepared by hanging drop using iPS cells retrieved after seven-day culture on a culture substrate (Design Example 5) in Example 2 and was then adhesively cultured, and show βIII tubulin, α-smooth muscle actin, and α-fetoprotein, respectively.

### DETAILED DESCRIPTION OF THE EMBODIMENTS

The following describes a culture method of the present invention and a culture substrate used in the culture method, which are optimally designed for rapid growth of iPS cells.

The inventors of the present invention have found that the rapid growth of iPS cells can be achieved by, in designing a hydrogel substrate for culturing iPS cells, optimizing the surface elastic modulus of the gel and optimizing the density of cell adhesion proteins immobilized on the gel surface, thereby accomplishing the present invention.

In the present invention, a cell culture substrate is used in which cell adhesion proteins are immobilized at a certain density on a surface of an elasticity-variable gel having a certain surface elastic modulus.

### [Elasticity-Variable Gel]

As the elasticity-variable gel, a gel can be used which includes, for example, a biopolymer such as collagen, gelatin, chitin, chitosan, alginic acid, and hyaluronic acid, and a synthetic polymer such as polyacrylamide, polydimethylsiloxane, and poly(ethylene glycol) diacrylate, and is formed by giving an appropriate cell adhesion property and controlling a degree of cross-linking.

The degree of cross-linking can be controlled by adjusting the concentration of a cross-linking agent, the reaction temperature, the reaction time, etc. When photoreaction is utilized for the cross-linking reaction, the degree of cross-linking can also be controlled by adjusting the intensity of irradiated light and the irradiation time, whereby the elastic modulus of the gel can be designed.

The elasticity-variable gel has an elastic modulus larger than 1 kPa and smaller than 100 MPa, preferably 10 kPa or larger and smaller than 10 MPa, and more preferably 10 kPa or larger and smaller than 1 MPa. For example, it is preferable to use a gel having an elastic modulus of 30 kPa, 40 kPa, 50 kPa, 60 kPa, 70 kPa, 80 kPa, or 90 kPa. In a case of a photocurable gelatin gel described below, its elastic modulus is suitably larger than 20 kPa and smaller than 100 kPa, preferably 30 kPa or larger and 90 kPa or smaller, and more preferably 40 kPa or larger and 60 kPa or smaller.

The use of such a gel having an elastic modulus of the above-stated numerical ranges can achieve far better rapid growth of iPS cells than growth of iPS cells in the culture system using existing Matrigel (registered trademark by Becton, Dickinson and Company), while their sternness such as undifferentiation property is maintained. It is not preferable to use a gel having an elastic modulus out of the above-stated numerical ranges, because such a case provides lower growth and culture behavior suggesting differentiation.

A gelatin gel, in particular a gel of a photocurable gelatin can suitably be used as the elasticity-variable gel in the present invention. To provide a gelatin with photocurability, photoreactive moieties are introduced to functional groups of various amino-acid residues in the gelatin via appropriate chemical bonds. Examples of the photoreactive moieties (also called functional groups with photoreactivity) include ethylenically unsaturated groups such as vinyl groups, allyl groups, styryl groups, and (meth)acryloyl groups. For example, a gelatin with styryl groups introduced (called a photocurable styrenated gelatin) can be used.

A gelatin, selected as a constituent in a cell culture material, can be dissolved at high concentration in water. For this reason, a gel of the gelatin can have a larger mechanical strength than that of a gel of another material, and the mechanical strength can be adjusted more widely. In addition, gelatin can have various molecular weights and is available at low cost, thereby providing advantages such as cost reduction in cell culture. Among ethylenically unsaturated groups to be introduced to the gelatin, styryl groups are particularly high in hydrophobicity and therefore are associated in an aqueous solution.

For this reason, the polymerization efficiency is higher in photopolymerization of the gelatin with styryl groups introduced than with another unsaturated groups introduced, which can reduce the curing time.

The photocurable gelatin gel can be obtained by preparing an aqueous solution containing a photocurable gelatin and a photoradical polymerization initiator and emitting light to the solution.

The photocurable gelatin can be obtained by reacting a gelatin with, for example, a compound (for example, 4-vinylbenzoic acid) having styryl group(s) and carboxy group(s) as photoreactive moieties in the presence of a carbodiimide as a condensing agent.

In this reaction, amino groups of proteins in the gelatin are reacted with the carboxy groups of the compound having the photoreactive moieties to form amide bonds, whereby the photoreactive moieties (for example, styryl groups) are introduced to the proteins in the gelatin. Examples of the carbodiimide used as the condensing agent in introducing the photoreactive moieties to the gelatin include dicyclohexylcarbodiimide (DCC), diethylcarbodiimide, diisopropylcarbodiimide (DIC), ethylcyclohexylcarbodiimide, diphenylcarbodiimide, 1-ethyl-3-(3-dimethylaminopropyl) carbodiimide hydrochloride (EDC), and 1-cyclohexyl-3-(3-dimethylaminopropyl) carbodiimide hydrochloride.

The compound to provide the gelatin with photocrosslinkability (photocurability) in the reaction above can be any compound as long as it includes three factors: functional groups having photoreactivity, such as vinyl groups described above; a hydrophobic skeleton having a high association property in water; and functional groups usable for chemical bonding with the gelatin. Thus, the configuration of the photocurable gelatin is not limited to the styryl group-introduced gelatin described above as an example.

The gelatin used here preferably has a weight-average molecular weight of 95,000 to 105,000 (measured by HPLC, in terms of polystyrene). The rate of introduction of photoreactive moieties (for example, styryl groups) to the gelatin is preferably, for example, 80 to 100% with respect to the total number of amino groups in the gelatin.

The weight-average molecular weight of the gelatin and the rate of induction of photoreactive moieties within such numerical ranges can provide a gelatin gel having a mechanical strength suitable for culturing iPS cells and an elastic modulus as described above.

The photoradical polymerization initiator may be any compound as long as it generates radicals by light irradiation. Examples of the photoradical polymerization initiator include: carbonyl compounds such as camphorquinone, acetophenone, benzophenone, and dimethoxy phenylacetophenone, and their derivatives; sulfur compounds such as dithiocarbamate, salts of xanthic acid, and thiophenol, and their derivatives; peroxides such as benzoyl peroxide and butyl peroxide, and their derivative; azobis compounds such as azobisisobutyronitrile and esters of azobisisobutyric acid, and their derivatives; halides such as bromopropane and chloromethyl naphthalene, and their derivatives; azide compounds such as phenyl azide, and their derivatives; xanthene dyes such as rhodamine, erythrosine, fluorescein, and eosin, and their derivatives; and riboflavin and its derivative. Among these compounds, camphorquinone such as sulfonyl camphorquinone is a preferable photoradical polymerization initiator because its biogenic safety has been established.

The photoradical polymerization initiator may be a single compound of these compounds and a combination of two or more of these compounds.

The photoradical polymerization initiator is used at, for example, 0.01 to 10% by mass, preferably 0.1 to 3% by mass, with respect to the total mass of the photocurable gelatin.

The aqueous solution that contains the photocurable gelatin and the photoradical polymerization initiator may be any aqueous solution as long as iPS cells to be used can live in the solution. Examples of the aqueous solution include physiological saline such as Ringer's solution and Locke's solution, phosphate buffer, and balanced saline such as Tyrode's solution, Hank's solution, Earle's solution, and HEPES solution.

The concentration of the photocurable gelatin in the aqueous solution is preferably 20 to 50% by mass, for example 30% by mass, with respect to the total mass of the aqueous solution.

The aqueous solution may contain an additional nutritional composition necessary for iPS cells to be cultured to grow. Examples of such a nutritional composition include minerals such as Na, K, Ca, Mg, P, and Cl, amino acids, vitamins, carbohydrates, fats, and growth factors, which can be selected as appropriate.

A light source for light emission in curing the aqueous solution containing the photocurable gelatin and the photoradical polymerization initiator into gel can be, for example, a halogen lamp, a xenon lamp, an incandescent lamp, a mercury lamp, an excimer laser, and an argon ion laser. It is preferable to emit a light having a wavelength of 300 to 800 nm using such a light source. The light irradiation is performed until the aqueous solution turns into gel. The gelation generally takes about 0.5 to 5 minutes.

The aqueous solution containing the photocurable gelatin and the photoradical polymerization initiator will be cured into gel (gelate) by light irradiation in the following mechanism. First, the light irradiation causes the photoradical polymerization initiator to generate radicals. The radicals thus generated trigger radical polymerization of functional groups (for example, vinyl groups) having photoreactivity on the photocurable gelatin. The radical polymerization of the functional groups having photoreactivity initiated by the light irradiation causes gelatin to be cross-linked via the functional groups, which forms a network structure. The network structure swells with surrounding medium, which promotes gelation and thus forms gelatin gel. It is noted that the network structure described above can have not only the cross-linked structure of the functional groups having photoreactivity but also amide bonds of amino groups and carboxy groups in the gelatin.

While the photocurable gelatin is being cured from liquid into gel with the progress of the radical polymerization, the degree of swelling of the gel being formed decreases as the cross-link density in the gelatin increases. This means increase in the mechanical strength of the gel being formed.

Thus, the gel having a mechanical strength suitable for culturing iPS cells and an elastic modulus as described above can be prepared by adjusting the irradiation time of light (control on the amount of radicals to be generated), the additive amount of the photoradical polymerization initiator, the concentration of gelatin in the aqueous solution, the molecular weight of the gelatin, the rate of introduction of the functional groups having photoreactivity into the gelatin, etc.

### [Cell Adhesion Protein]

The cell adhesion proteins to be immobilized on the surface of the elasticity-variable gel are preferably laminins, fibronectins, and vitronectins, for example. Alternatively, it is possible to utilize for surface immobilization a peptide sequence in the molecular structure of these proteins that forms a particular specific binding with an integrin of the cell. Laminins are particularly preferable as the cell adhesion proteins.

Laminins are giant proteins in the basal lamina of extracellular matrix, and are heterotrimers including three subunit chains called α, β, and γ. There are 11 kinds of subunits: α1 to α5 for the α chain, β1 to β3 for the β chain, and γ1 to γ3 for the γ chain. The combination of subunit chains determines the name of a laminin. For example, a laminin having α1 for the subunit α, β1 for the subunit β, and γ1 for the subunit γ is called laminin-111. Thus, 45 kinds of laminin can theoretically exist according to the combinations of the subunits.

Examples of the laminin to be immobilized on the surface of the gelatin gel of the present invention include 19 kinds of laminin families (laminin-111, 121, 211, 221, 212,222, 213, 3A11, 3A21, 3A32, 3A33, 3B32, 411, 421, 423, 511, 521, 522, and 523), which are currently known, another kind of laminin that can be found from these laminins, refined products from organs and cellular secretions, and recombinant laminins

In the present invention, laminin-111 is particularly preferable.

### [Chemical Method of Immobilizing Cell Adhesion Proteins on Elasticity-Variable Gel Surface]

When a gel of a photocurable styrenated gelatin is used as the elasticity-variable gel and laminins are used as the cell adhesion proteins, for example, the two materials are reacted in the presence of a carbodiimide as a condensing agent. This chemically immobilizes the laminins on the surface of the gelatin via amide bonds by the reaction of carboxy groups on the gelatin gel surface and amino groups of the laminins

The carbodiimides recited above as usable in introducing photoreactive moieties to the gelatin can be used here.

It is noted that the present invention is also directed to a gel including a photocurable gelatin and a laminin immobilized on a surface of the gel, in particular, a gel in which the photocurable gelatin is a photocurable styrenated gelatin and/or the laminin is laminin-111.

The cell adhesion proteins (for example, laminins) are reacted with the elasticity-variable gel (for example, a gelatin gel) at a reacting concentration of 0.1 to 2 mg, preferably 0.5 to 1.0 mg, with respect to 1 mL of the elasticity-variable gel. Thus, far better rapid growth than growth in the culture system using existing Matrigel (registered trademark) can be achieved.

### [Induced Pluripotent Stem Cell]

The culture method of the present invention is assumed to be applied to induced pluripotent stem cells (iPS cells), embryonic stem cells (ES cells), or other kinds of stem cells. The present invention is particularly directed to iPS cells, which are capable of being differentiated into various types of cells and thus expected to be utilized in medical treatment.

In general, an iPS cell is prepared by introducing reprogramming factors (four genes: Oct3/4, Sox2, Klf4, and c-Myc) into an adult somatic cell.

The present invention may be applied to human iPS cells established directly from somatic cells of a patient for clinical use, and iPS cells available from cell banks for experimental works.

### [Dispersive Culture of iPS Cells]

As described above, conventional methods of culturing iPS cells include a passage culture using a feeder cell sheet and a passage culture using Matrigel (BD Matrigel™ Basement Membrane Matrix: BD 354234) instead of a feeder cell sheet.

The operation for culturing iPS cells in the present invention can be performed on a cell culture substrate including the above-described elasticity-variable gel with the adhesion proteins immobilized thereon, similarly to the normal culture operation on Matrigel. In this operation, iPS cells in single-cell state are dispersively seeded on the culture substrate according to the present invention to be cultured.

The following describes an exemplary method of dispersively culturing iPS cells on the cell culture substrate including the elasticity-variable gel of the present invention with adhesion proteins immobilized thereon.

A ROCK inhibitor is added at a final concentration of 10 iM to a culture dish of iPS cells cultured in advance to subconfluence, and the cells are incubated at 37° C. for one hour or more. The culture medium is then aspirated off, and Accutase (or 0.05% Trypsin-EDTA) as a reagent for cell retrieval is added at 1 mL per 6-mm dish. The cells are incubated at 37° C. for five to seven minutes. Subsequently, the dish is tapped, 3 mL of a feeder-free serum-free culture medium (mTeSR™ 1: STEMCELL Technologies 05850; when using 0.05% Trypsin-EDTA, a culture medium containing serum such as MEF medium) is added to the dish, and the cells are roughly dissociated into single cells by pipetting up and down. The cell suspension thus obtained is transferred into a 15-mL tube to be centrifuged at a centrifugal acceleration of 170 (×g) (1,000 rpm) for five minutes. The supernatant is aspirated off, the resultant is suspended with a feeder-free serum-free culture medium, and the cells are seeded on a culture substrate of the laminin-immobilized elasticity-variable gelatin gel of the present invention. A ROCK inhibitor is added for a final concentration of 10 miM. From the following day, adhesion of a cell cluster is observed, and the culture medium is replaced once per day, thereby continuing the culture.

The iPS cells, dispersively cultured on the culture substrate of the elasticity-variable gel of the present invention with the adhesion proteins immobilized thereon, can healthily grow to a density of 1.5×10⁵ cells/cm² or more in a culture duration of about seven days, maintaining their undifferentiation property and multipotency. Examples

The following describes features of the present invention more specifically by means of examples and a comparative example. Materials, usage, proportions, processes, and procedures described in the following examples can be modified as appropriate without departing from the spirit and scope of the present invention. Thus, the following examples are not intended to restrict interpretation of the scope of the invention.

### [Production Example 1: Preparation of Elasticity-Variable Hydrogel]

A visible light having a wavelength of around 500 nm was emitted to a sol (containing 1.5% by mass of sulfonyl camphorquinone) of 30% by mass of a photocurable gelatin (photocurable styrenated gelatin: StG) with vinyl groups introduced (the molecular weight of the gelatin: about 100,000, the rate of introduction of the vinyl groups: 98% with respect to amino groups in the gelatin) in phosphate buffered saline (hereinafter called PBS) to cause photocrosslinking by way of radical polymerization of the vinyl groups, thereby a gel was prepared. The irradiation time of light was changed between 100 to 800 seconds to prepared gelatin gels. The surface elastic moduli of the prepared gelatin gels were measured by a microscopic injection test using an atomic force microscope. FIG. 1 illustrates change in surface elastic moduli (Young's moduli) of the gels corresponding to the respective irradiation times of light in the gel preparation.

As illustrated in FIG. 1, the irradiation time of light of 300 seconds provided a gelatin gel having a surface elastic modulus of 50 kPa. The irradiation times of light of 300 seconds or more provided a linear increase in Young's moduli.

### [Production Example 2: Immobilization of Laminin on Elasticity-Variable Gel Surface]

On a surface of the gel having the surface elastic modulus of 50 kPa, which was prepared in Production Example 1, a solution of 0.5 mg/mL of 1-ethyl-3-(3-dimethylaminopropyl) carbodiimide hydrochloride (water-soluble carbodiimide) in phosphate buffer (pH 4.8) was reacted overnight at 4° C. Subsequently, solutions of fluorescently-labeled laminin-111 in phosphate buffer at various concentrations were dropped on the gel to be reacted at 4° C. for a certain time.

### [Evaluation of the Amount of Laminins on the Gel Surface]

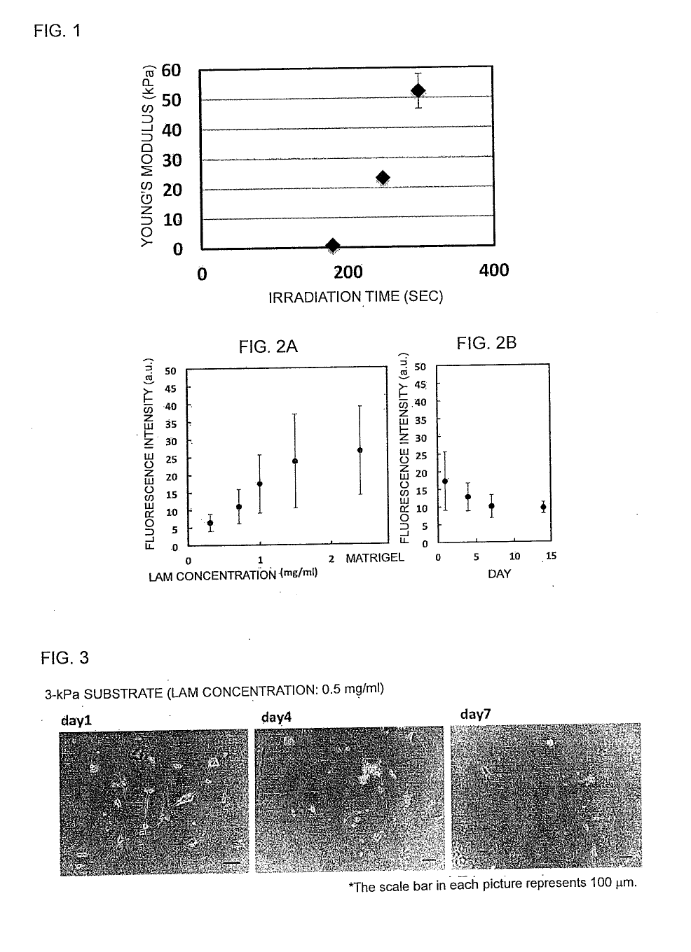

After the reactant obtained in Production Example 2 was washed with PBS, immunostaining was performed for laminin-111 (hereinafter called LAM) to perform fluorescence observation of the gel surface using a confocal microscope or an electron-multiplier CCD camera. FIG. 2A illustrates change in fluorescence intensity with respect to the concentrations of reacted LAM, and FIG. 2B illustrates change in fluorescence intensity in 14 days after the LAM immobilization (the LAM concentration: 1.0 mg/ml). It is noted that the fluorescence intensity illustrated in FIG. 2 corresponds to the amount of LAM immobilized on the gel surface (immobilization density).

The amounts of LAM immobilized on the substrate were roughly constant among the reaction times of one hour or more. In contrast, linear increase in accordance with the increase in the LAM reacting concentration was shown. In particular, the reaction condition with the LAM concentration of 1.5 mg/mL achieved an amount of laminin-111 immobilized on the gelatin gel equivalent to the amount in an existing culture system Matrigel (registered trademark) of Becton, Dickinson and Company (FIG. 2A).

Furthermore, temporal stability of immobilized LAM (desorption of LAM) was evaluated by way of fluorescent observation of the gel surface at certain points of time after the immobilization. Although slight decrease was observed in fluorescence intensity due to desorption of non-specifically adhesive LAM, a constant amount of LAM was stably present on the gel surface up to 14 days after the immobilization (FIG. 2B).

### [Design Example 1]

According to the procedures in Production Example 1 and Production Example 2 described above, gelatin gels were prepared so as to have a surface elastic modulus of 3 kPa with a controlled irradiation time of light. Immobilizing reactions were performed on the gelatin gels at respective LAM reacting concentrations of 0.1, 0.25, 0.5, 1.0, and 1.5 mg/mL, thereby culture substrates were obtained which had laminin-immobilization densities linearly adjusted as illustrated in FIG. 2A (also called 3-kPa substrates).

### [Design Example 2]

According to the procedures in Production Example 1 and Production Example 2 described above, gelatin gels were prepared so as to have a surface elastic modulus of 20 kPa with a controlled irradiation time of light. Immobilizing reactions were performed on the gelatin gels at respective LAM reacting concentrations of 0.1, 0.25, 0.5, 1.0, and 1.5 mg/mL, thereby culture substrates were obtained which had laminin-immobilization densities linearly adjusted as illustrated in FIG. 2A (also called 20-kPa substrates).

### [Design Example 3]

According to the procedures in Production Example 1 and Production Example 2 described above, gelatin gels were prepared so as to have a surface elastic modulus of 50 kPa with a controlled irradiation time of light. Immobilizing reactions were performed on the gelatin gels at respective LAM reacting concentrations of 0.1, 0.25, 0.5, 1.0, and 1.5 mg/mL, thereby culture substrates were obtained which had laminin-immobilization densities linearly adjusted as illustrated in FIG. 2A (also called 50-kPa substrates).

### [Design Example 4]

According to the procedures in Production Example 1 and Production Example 2 described above, gelatin gels were prepared so as to have a surface elastic modulus of 100 kPa with a controlled irradiation time of light. Immobilizing reactions were performed on the gelatin gels at respective LAM reacting concentrations of 0.1, 0.25, 0.5, 1.0, and 1.5 mg/mL, thereby culture substrates were obtained which had laminin-immobilization densities linearly adjusted as illustrated in FIG. 2A (also called 100-kPa substrates).

### [Substrate for Comparative Example]

Matrigel (registered trademark) of Becton, Dickinson and Company was melted on ice, and then diluted 30 fold with a serum-free medium. Two milliliters of the diluted Matrigel was transferred into a 60-mm dish to be incubated for one hour at 37° C. (or overnight at 4° C.). The diluted Matrigel was aspirated off, and the dish was lightly washed with a feeder-free serum-free medium, thereby a Matigel substrate was obtained.

### Example 1

### [Evaluation of Growth Behavior of iPS Cells on Substrates in Design Examples 1 to 4 and Comparative Example]

The present example evaluated the growth behavior of iPS cells on each of the substrates described above.

A human iPS cell strain 253G1 was dispersively seeded at 2.0×10⁴ cells/cm² on the substrates prepared in Design Examples 1 to 4 described above and the substrate in Comparative Example to be cultured for seven days. The density of adherent cells was measured on each day. For comparison, the human iPS cell strain was dispersively seeded and cultured in a similar way on substrates without immobilized LAM in Design Examples 1 to 4 described above.

FIGS. 3 to 7 show phase-contrast micrographs of the respective substrates having the LAM reacting concentration of 0.5 mg/mL on a first day (day1), fourth day (day4), and seventh day (day7). FIGS. 8A to 8D show change in density of immobilized cells on each substrate at different LAM-reaction concentrations with respect to culturing days. FIGS. 8A to 8D show the results of the 3-kPa substrates, the 20-kPa substrates, the 50-kPa substrates and the Matrigel substrate, and the 100-kPa substrates and the Matrigel substrate, respectively.

As for the 3-kPa substrates prepared in Design Example 1, the number of cells decreased with the culturing days under any laminin-concentration condition (see FIG. 3 and FIG. 8A).

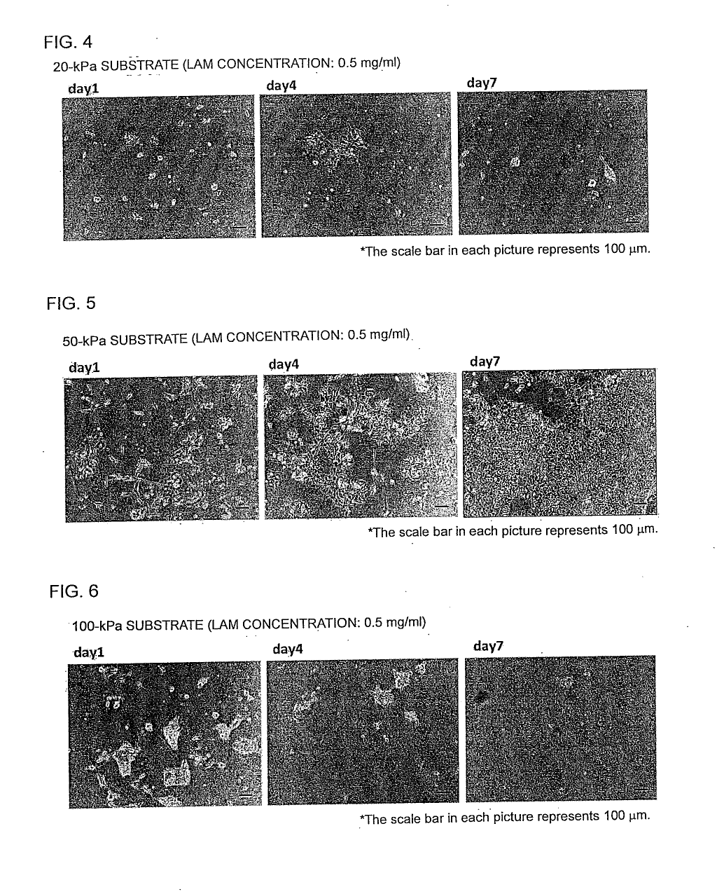

As for the 20-kPa substrates prepared in Design Example 2, small cell clusters were formed under any laminin-concentration condition. The cell clusters were observed to collapse thereafter into single cells, and no growth of the cells was observed (see FIG. 4 and FIG. 8B).

In contrast, as for the 50-kPa substrates prepared in Design Example 3, growth of iPS cells was observed under the LAM reacting concentration of 0.5 mg/mL or 1.0 mg/mL (see FIG. 5 and FIG. 8C).

As for the 100-kPa substrates prepared in Design Example 4, growth of the iPS cells was observed under the LAM reacting concentration of 0.1 mg/mL while no growth was observed under the other LAM reacting concentrations (see FIG. 6 and FIG. 8D).

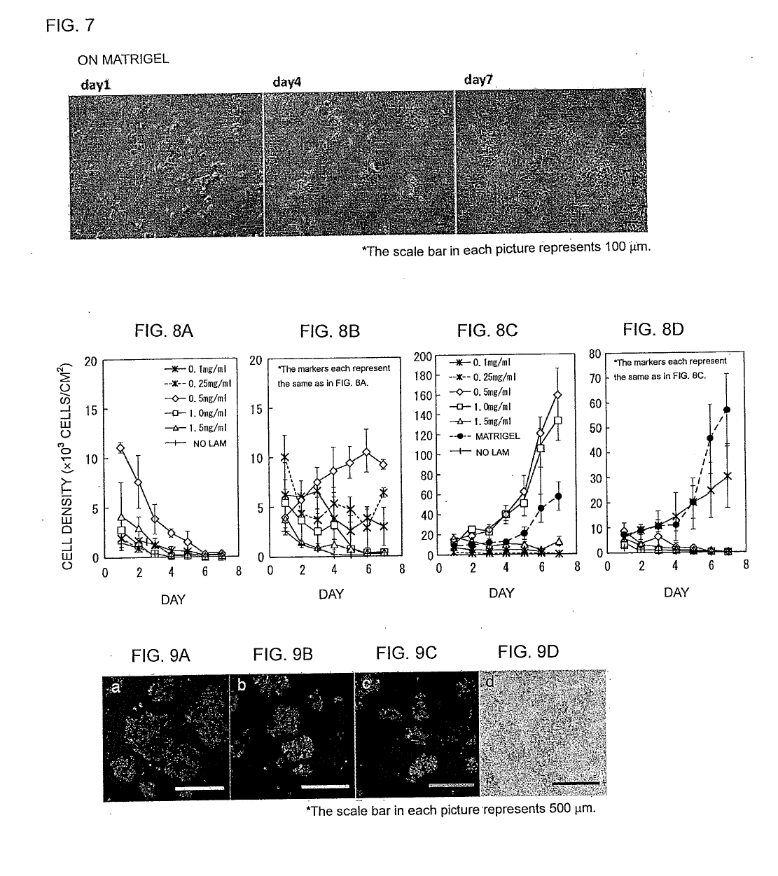

As for the Matrigel substrate in Comparative Example, the cells moderately grew for seven days after the seeding (see FIG. 7).

With reference to FIG. 8C in particular, a result shows that far better cell growth was observed on the 50-kPa substrates in Design Example 3 under the LAM reacting concentrations of 0.5 mg/mL and 1.0 mg/mL than on the Matrigel substrate in Comparative Example. However, a cell cluster was formed but no growth was observed on the 50-kPa substrate under the LAM reacting concentration of 1.5 mg/mL, which provided an amount of immobilized laminins equivalent to that of Matrigel (see FIG. 2).

The results described above show that there is an optimal condition for growth of iPS cells, which includes optimized parameters of the surface elastic modulus of the culture substrate and the surface density of immobilized laminins. The present invention provides a substrate for rapid growth of iPS cells by optimally designing both of the parameters.

### Example 2

[Evaluation of Stemness of iPS Cells Retrieved after Cultured]

The present example evaluated the iPS cells cultured under the substrate conditions in which cell growth was observed to determine whether they maintained normal stemness (self-replicating growth potential, undifferentiation property, and multipotency).

According to the procedures in Production Example 1 and Production Example 2 described above, a gelatin gel was prepared so as to have a surface elastic modulus of 50 kPa with a controlled irradiation time of light. Immobilizing reaction was performed on the gelatin gel at an LAM reacting concentration of 0.5 mg/mL, thereby a culture substrate (Design Example 5) to be used here was obtained. According to the procedure in Example 1 described above, a human iPS cell strain was seeded on the substrate in Design Example 5 to be cultured for seven days. The iPS cells cultured for seven days were retrieved from the culture substrate to be reseeded on mouse embryonic fibroblasts (MEFs) as feeder cells. After seven-day culture, immunostaining was performed to examine expression of undifferentiation marker proteins (SSEA-1, SSEA-4, TRA-1-60, and TRA-1-81). FIGS. 9A to 9D show images of immunofluorescent-stained undifferentiation marker proteins observed in the cultured iPS cells. FIGS. 9A to 9D show SSEA-4, TRA-1-60, TRA-1-81, and SSEA-1, respectively.

As shown in FIGS. 9A to 9D, normal colonization of the iPS cells, positive reaction in all of the three positive markers (SSEA-4, TRA-1-60, and TRA-1-81), and negative reaction in the negative marker (SSEA-1) were confirmed. Thus, it was confirmed that the undifferentiation property was maintained in the iPS cells that were retrieved after being cultured by the culture method of the present invention.

Furthermore, regarding the mutipotency of the retrieved iPS cells, an embryoid prepared by hanging drop was adhesively cultured for natural differentiation induction.

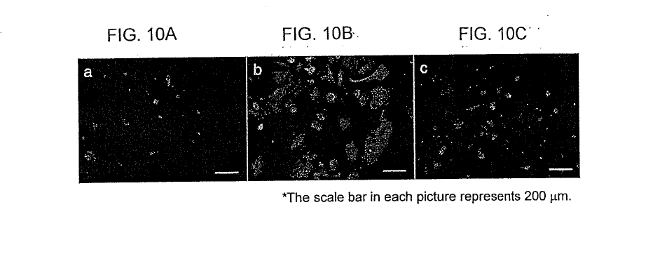

Immunostaining was performed on the iPS cells retrieved by the procedure described above to examine expression of triploblastic differentiation marker proteins (βIII tubulin, α-smooth muscle actin, and α-fetoprotein). The result was expressions of all of the makers, and it was confirmed that the multipotency was retained. FIGS. 10A to 10C show images of immunofluorescent-stained differentiation marker proteins. FIGS. 10A to 10C show βIII tubulin, α-smooth muscle actin, and α-fetoprotein, respectively.

As described above, the retrieved iPS cells exhibited normal behaviors in self-replicating growth potential, undifferentiation property, and multipotency. Thus, it was confirmed that the iPS cells cultured by the cultured method of the present invention retained sternness.

1. A method of culturing an induced pluripotent stem cell, the method comprising:
using a cell culture substrate including an elasticity-variable gel having an elastic modulus larger than 1 kPa and smaller than 100 MPa and a cell adhesion protein immobilized on a surface of the elasticity-variable gel.

2. The culture method according to claim 1, wherein the elasticity- variable gel is a gel of a photocurable gelatin.

3. The culture method according to claim 2, wherein the photocurable gelatin is a photocurable styrenated gelatin.

4. The culture method according to claim 1, wherein the cell adhesion protein is a laminin.

5. The culture method according to claim 4, wherein the laminin is laminin-111.

6. The culture method according to claim 1, wherein the cell adhesion protein is immobilized at a reacting concentration of 0.1 mg to 2.0 mg with respect to 1 mL of the elasticity-variable gel.

7. The culture method according to claim 1, wherein the induced pluripotent stem cell is a human induced pluripotent stem cell.

8. A culture substrate for an induced pluripotent stem cell, the culture substrate comprising:
an elasticity-variable gel having an elastic modulus larger than 1 kPa and smaller than 100 MPa; and
a cell adhesion protein immobilized on a surface of the elasticity-variable gel.

9. The culture substrate according to claim 8, wherein the elasticity-variable gel is a gel of a photocurable gelatin.

10. The culture substrate according to claim 8, wherein the photocurable gelatin is a photocurable styrenated gelatin.

11. The culture substrate according to claim 8, wherein the cell adhesion protein is a laminin.

12. The culture substrate according to claim 11, wherein the laminin is laminin-111.

13. The culture substrate according to claim 8, wherein the cell adhesion protein is immobilized at a reacting concentration of 0.1 mg to 2.0 mg with respect to 1 mL of the elasticity-variable gel.

14. The culture substrate according to claim 8, wherein the induced pluripotent stem cell is a human induced pluripotent stem cell.

15. A gel comprising:
a photocurable gelatin; and
a laminin immobilized on a surface of the gel.

16. The gel according to claim 15, wherein the photocurable gelatin is a photocurable styrenated gelatin.

17. The gel according to claim 15, wherein the laminin is laminin-111.
